(19) United States
(12) Patent Application Publication
(10) Pub. No.: US 2012/0095354 A1
(43) Pub. Date: Apr. 19, 2012

(54) QUANTITATIVE IMAGING WITH MULTI-EXPOSURE SPECKLE IMAGING (MESI)

(76) Inventors: Andrew Dunn, Austin, TX (US); Ashwin B. Parthasarathy, Austin, TX (US); William James Tom, Houston, TX (US)

(21) Appl. No.: 13/211,962

(22) Filed: Aug. 17, 2011

Related U.S. Application Data
(63) Continuation-in-part of application No. PCT/US2010/024427, filed on Feb. 17, 2010.
(60) Provisional application No. 61/153,004, filed on Feb. 17, 2009.

(51) Int. Cl. A61B 5/0265

(52) U.S. Cl. 600/504

(57) ABSTRACT

Methods and systems relating to multi-exposure laser speckle contrast imaging are provided. One such system comprises a laser light source, a light modulator, and a detector for the measurement of reflected light comprising at least one camera, at least one magnification objective, and at least one microprocessor or data acquisition unit.

### CROSS-REFERENCE TO RELATED APPLICATIONS

This application is a continuation-in-part of PCT/US2010/024427 filed Feb. 17, 2010 and claims priority to U.S. patent application Ser. No. 61/153,004 filed Feb. 17, 2009, which is incorporated herein by reference.

### STATEMENT OF GOVERNMENT INTEREST

This invention was made with government support under Grant Nos. CBET-0644638 and CBET/0737731 awarded by the National Science Foundation and under Grant No. 0735136N awarded by the American Heart Association. The government has certain rights in the invention.

### BACKGROUND

Laser Speckle Contrast Imaging (LSCI) is a popular optical technique to image blood flow. It was introduced by Fercher and Briers in 1981, and has since been used to image blood flow in the brain, skin and retina. Since LSCI is a full field imaging technique, its spatial resolution is not at the expense of scanning time unlike more traditional flow measurement techniques like scanning Laser Doppler Imaging (LDI). For these reasons LSCI has been used to quantify the cerebral blood flow (CBF) changes in stroke models and for functional activation studies.

The advantages of LSCI have created considerable interest in its application to the study of blood perfusion in tissues such as the retina and the cerebral cortices. In particular, functional activation and spreading depolarizations in the cerebral cortices have been explored using LSCI. The high spatial and temporal resolution capabilities of LSCI are incredibly useful for the study of surface perfusion in the cerebral cortices because perfusion varies between small regions of space and over short intervals of time.

One criticism of LSCI is that it can produce good measures of relative flow but cannot measure baseline flows. This has prevented comparisons of LSCI measurements to be carried out across animals or species and across different studies. Lack of baseline measures also make calibration difficult. This limitation has been attributed to the use of an approximate model for measurements. Another limitation of LSCI, especially for imaging cerebral blood flow, has been the inability of traditional speckle models to predict accurate flows in the presence of light scattered from static tissue elements. Traditionally this problem has been avoided in imaging cerebral blood flow by performing a full craniotomy (removal of skull). Such a procedure is traumatic and can disturb normal physiological conditions. Imaging through an intact yet thinned skull can drastically improve experimental conditions by being less traumatic, reducing the impact of surgery on normal physiological conditions and enabling chronic and long term studies. One of the advantages of imaging CBF in mice is that LSCI can be performed through an intact skull. However variations in skull thickness lead to significant variability in speckle contrast values.

### SUMMARY

The present disclosure generally relates to imaging blood flow, and more specifically, to quantitative imaging with multi-exposure speckle imaging (MESI).

In certain embodiments, the present disclosure provides a MESI system comprising: a laser light source for the illumination of a sample; a light modulator; and a detector for the measurement of reflected light comprising at least one camera, at least one magnification objective, and at least one microprocessor or data acquisition computer.

In some embodiments, the present disclosure also provides methods for quantitative blood flow imaging that comprise: providing a MESI system comprising a laser light source for the illumination of a sample; a light modulator; and a detector for the measurement of reflected light comprising at least one camera, at least one magnification objective, and at least one microprocessor or data acquisition computer; illuminating a sample and detecting a speckle pattern using the MESI system; and computing a quantitative blood flow image. In some embodiments, a quantitative blood flow image may be computed using a speckle model of the present disclosure.

### DRAWINGS

Some specific example embodiments of the disclosure may be understood by referring, in part, to the following description and the accompanying drawings.

FIG. 1A shows a schematic of a multi-exposure speckle imaging (MESI) system, according to one embodiment.

FIG. 1B is a speckle contrast image at 0.1 ms exposure duration obtained by a MESI system of the present disclosure.

FIG. 1C is a speckle contrast image at 5 ms exposure duration obtained by a MESI system of the present disclosure.

FIG. 1D is a speckle contrast image at 40 ms exposure duration (scale bar=50 μm) obtained by a MESI system of the present disclosure.

FIG. 2A depicts a cross-section of a microfluidic flow phantom (not to scale) without a static scattering layer. The sample was imaged from the top.

FIG. 2B depicts a cross-section of microfluidic flow phantom (not to scale) with a static scattering layer. The sample was imaged from the top.

FIG. 3 is a graph depicting the Multi-Exposure Speckle Contrast data fit to the speckle model of the present disclosure. Speckle variance as a function of exposure duration is shown for different speeds. Measurements were made on a sample with no static scattering layer (FIG. 2A).

FIG. 4 is a graph depicting the Multi-Exposure Speckle Contrast data analyzed by spatial (ensemble) sampling (solid lines) and temporal (time) sampling (dotted lines). Measurements were made at 2 mm/sec. The three curves for each analysis technique represent different amounts of static scattering. μ′ₛ values refer to the reduced scattering coefficient in the 200 μm static scattering layer. μ′ₛ=0 cm⁻¹: No static scattering layer (FIG. 2A), μ′ₛ=4 cm⁻¹: 0.9 mg/g of TiO₂ in static scattering layer (FIG. 2B), μ′ₛ=8 cm⁻¹: 1.8 mg/g of TiO₂ in static scattering layer (FIG. 2B).

FIG. 5 is a graph depicting the Multi-Exposure Speckle Contrast data from two samples fit to the speckle model of the present disclosure. Speckle variance as a function of exposure duration is shown for two different speeds and two levels of static scattering. Solid lines represent measurements made on sample without static scattering layer. Dotted lines represent measurements made on sample with static scattering layer. μ′ₛ values refer to the reduced scattering coefficient in the 200 μm static scattering layer. μ′ₛ=0 cm⁻¹: No static scattering layer (FIG. 2A), μ′ₛ=8 cm⁻¹: 1.8 mg/g of TiO₂ in static scattering layer (FIG. 2B).

FIG. 6 is a graph depicting the percentage deviation in τ_{c} over changes in amount of static scattering for different speeds (estimated using Equation 4). Data from all three static scattering cases μ′ₛ=0 cm⁻¹: No static scattering layer (FIG. 2A), μ′ₛ=4 cm⁻¹: 0.9 mg/g of TiO₂ in static scattering layer (FIG. 2B), μ′ₛ=8 cm⁻¹: 1.8 mg/g of TiO₂ in static scattering layer (FIG. 2B) was used in this analysis.

FIG. 7 is a graph depicting the performance of different models to relative flow. Baseline speed: 2 mm/sec. Plot of relative τ_{c}, to relative speed. Plot should ideally be a straight line (dashed line). Multi-Exposure estimates extend linear range of relative τ_{c}, estimates. Error bars indicate standard error in relative correlation time estimates. Measurements were made using a microfluidic phantom with no static scattering layer (FIG. 2A).

FIG. 8A is a graph that quantifies the effect of static scattering on relative τ_{c} measurements. Plot of relative correlation time (Equation 12) to relative speed. Baseline Speed—2 mm/sec. The three curves represent different amounts of static scattering. μ′ₛ values refer to the reduced scattering coefficient in the 200 μm static scattering layer. μ′ₛ=0 cm⁻¹: No static scattering layer (FIG. 2A), μ′ₛ=4 cm⁻¹: 0.9 mg/g of TiO₂ in static scattering layer (FIG. 2B), μ′ₛ=8 cm⁻¹: 1.8 mg/g of TiO₂ in static scattering layer (FIG. 2B).

FIG. 8B is a graph that quantifies the effect of static scattering on relative τ_{c} measurements. Plot of relative correlation time (Equation 12) to relative speed. Baseline Speed—2 mm/sec. The three curves represent different amounts of static scattering. Error bars indicate standard error in estimates of relative correlation times. μ′ₛ values refer to the reduced scattering coefficient in the 200 μm static scattering layer. μ′ₛ=0 cm⁻¹: No static scattering layer (FIG. 2A), μ′ₛ=4 cm⁻¹: 0.9 mg/g of TiO₂ in static scattering layer (FIG. 2B), μ′ₛ=8 cm⁻¹: 1.8 mg/g of TiO₂ in static scattering layer (FIG. 2B).

FIG. 9A shows a schematic of a MESI system according to one embodiment.

FIG. 9B are speckle contrast images of mouse cortex obtained at various camera exposure durations using a MESI system.

FIG. 10A is a speckle contrast image (5 ms exposure duration) illustrating the partial craniotomy model. The regions within the closed loops (Regions 1, 3 and 5) are in the craniotomy. Regions outside the closed loops (Regions 2, 4 and 6) are in the thin skull region.

FIG. 10B is a speckle Contrast image of a branch of the MCA, illustrating ischemic stroke induced using photo thrombosis before stroke.

FIG. 10C is a speckle Contrast image of a branch of the MCA, illustrating ischemic stroke induced using photo thrombosis after stroke.

FIG. 11A is a speckle contrast image (5 ms exposure) illustrating regions of different flow.

FIG. 11B is a time integrated speckle variance curves with decay rates corresponding to flow rates. The data points have been fit to Equation 11.

FIG. 12A is an illustration of partial craniotomy model. The regions enclosed by the closed loops (regions 1, 3 & 5) are located in the craniotomy. Regions outside of the closed loops (regions 2, 4 & 6) are located in the thinned (but intact) skull.

FIG. 12B is a time integrated speckle variance curves illustrating the influence of static scattering due to the presence of the thinned skull. A decrease in the value of ρ indicates an increase in the amount of static scattering. Regions 2 and 4 show distinct offset at large exposure durations. This offset it due to increased vₛ over the thinned skull.

FIG. 13A is a graph depicting the time course of relative blood flow change in Region 1 in FIG. 12A as estimated using a MESI technique. The flow estimates in first 10 minutes were considered as baseline. The reduction in blood flow due to the stroke, is estimated to be ˜100%, which indicates that blood supply to the artery has been completely shut off.

FIG. 13B depicts MESI curves illustrating the change in the shape of the curve as blood flow decreases. The MESI curve obtained after the stroke is found to be similar in shape to that obtained after the animal was sacrificed. This is a qualitative validation of ˜100% decrease in blood flow in the artery.

FIG. 14A is a graph depicting relative blood flow changes estimated using a MESI technique in 3 pairs of regions across the boundary (FIG. 12A). The change in blood flow is found to be similar for each pair of regions.

FIG. 14B is a graph depicting the relative blood flow changes estimated using the LSCI technique (at 5 ms exposure) in 3 pairs of regions across the boundary (FIG. 12A). The change in blood flow is not similar for each pair of regions. This difference is especially prominent over the vessel (Regions 1 and 2).

FIG. 15A is a full field relative correlation time map obtained using the methods of the present disclosure.

FIG. 15B is a full field relative correlation time map obtained using LSCI technique (5 ms exposure). The boundary (corresponding to the boundary between the thin skull and the craniotomy) indicated by the red arrow is clearly visible in (b), but not in (a). There is a clear change gradient in the region indicated by the star in (b), but this gradient is invisible in (a). The vessel circled is more visible in (a) compared to (b).

FIG. 16 is a graph depicting the comparison of the percentage reduction in blood flow obtained in regions 1 and 2 (FIG. 12A) using the present disclosure with two different speckle expressions (Lorentzian: Equation 11 and Gaussian: Equation 13) and multiple single exposure LSCI estimates.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are described in more detail below. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure generally relates to imaging blood flow, and more specifically, to quantitative imaging with multi-exposure speckle imaging (MESI).

LSCI is a minimally invasive full field optical technique used to generate blood flow maps with high spatial and temporal resolution. The lack of quantitative accuracy and the inability to predict flows in the presence of static scatterers, such as an intact or thinned skull, have been the primary limitation of LSCI. Accordingly, in one embodiment, the present disclosure provides a Multi-Exposure Speckle Imaging (MESI) system that has the ability to obtain quantitative baseline flow measures. Similarly, in another embodiment, the present disclosure also provides a speckle model that can discriminate flows in the presence of static scatters. In some embodiments, the speckle model of the present disclosure, along with a MESI system of the present disclosure, in the presence of static scatterers, can predict correlation times of flow consistently to within 10% of the value without static scatterers compared to an average deviation of more than 100% from the value without static scatterers using traditional LSCI. The details of a MESI system and speckle model of the present disclosure will be discussed in more detail below.

In general, speckle arises from the random interference of coherent light. When collecting laser speckle contrast images, coherent light is used to illuminate a sample and a photodetector is then used to receive light that has scattered from varying positions within the sample. The light will have traveled a distribution of distances, resulting in constructive and destructive interference that varies with the arrangement of the scattering particles with respect to the photodetector. When this scattered light is imaged onto a camera, it produces a randomly varying intensity pattern known as speckle. If scattering particles are moving, this will cause fluctuations in the interference, which will appear as intensity variations at the photodetector. The temporal and spatial statistics of this speckle pattern provide information about the motion of the scattering particles. The motion can be quantified by measuring and analyzing temporal variations and/or spatial variations.

Using the latter approach, 2-D maps of blood flow can be obtained with very high spatial and temporal resolution by imaging the speckle pattern onto a camera and quantifying the spatial blurring of the speckle pattern that results from blood flow. In areas of increased blood flow, the intensity fluctuations of the speckle pattern are more rapid, and when integrated over the camera exposure time (typically 1 to 10 ms), the speckle pattern becomes blurred in these areas. By acquiring a raw image of the speckle pattern and quantifying the blurring of the speckles in the raw speckle image by measuring the spatial contrast of the intensity variations, spatial maps of relative blood flow can be obtained. To quantify the blurring of the speckles, the speckle contrast (K) is calculated over a window (usually 7×7 pixels) of the image as,

$$K = \frac{{σ}_{s}}{〈 I 〉} , \qquad Equation 1$$

where σₛ is the standard deviation and <I> is the mean of the pixels of the window. For slower speeds, the pixels decorrelate less and hence K is large and vice versa.

Although speckle contrast values are indicative of the level of motion in a sample, they are not directly proportional to speed or flow. To obtain quantitative blood flow measurements from speckle contrast values, two steps are typically performed. The first step is to accurately relate the speckle contrast values, which are obtained from a time-integrated measure of the speckle intensity fluctuations using Equation 1 above, to a speckle correlation time (τ_{c}). The second step is to relate the speckle correlation time to the underlying flow or speed.

The relationship between speckle contrast values, K, and speckle correlation time, τ_{c}, is rooted in the field of dynamic light scattering (DLS). The correlation time of speckles is the characteristic decay time of the speckle decorrelation function. The speckle correlation function is a function that describes the dynamics of the system using backscattered coherent light. Under conditions of single scattering, small scattering angles and strong tissue scattering, the correlation time can be shown to be inversely proportional to the mean translational velocity of the scatterers. Strictly speaking this assumption that τ_{c}∝1/v (where v is the mean velocity) is most appropriate for capillaries where a photon is more likely to scatter of only one moving particle and succeeding phase shifts of photons are totally independent of earlier ones. Hence great care should be observed when using this expression. The measurements in the present disclosure are made in channels that mimic smaller blood vessels and hence this relation between the correlation time and velocity can be used.

The uncertainty over the relation between correlation time and velocity is a fundamental limitation for all DLS based flow measurement techniques. Nevertheless, quantitative flow measurements can be performed through accurate estimation of the correlation times. The correlation times can be related to velocities through external calibration. The speckle contrast can be expressed in terms of the correlation time of speckles and the exposure duration of the camera. The MESI system of the present disclosure obtains speckle images at different exposure durations and uses this multi-exposure data to quantify τ_{c}. Previous efforts to obtain speckle images at multiple exposure durations have been limited to a few durations or to line scan cameras.

In one embodiment, the present disclosure provides a MESI system that is able to obtain images over a wide range of exposure durations (50 μs to 80 ms). Accordingly, a MESI system of the present disclosure is able to obtain better estimates of correlation times of speckles.

A. Speckle Model

Speckle contrast has been related to the exposure duration of a camera and correlation time of the speckles using the theory of correlation functions and time integrated speckle. The theory of correlation functions has been widely used in dynamic light scattering (DLS) and LSCI is a direct extension of it. The temporal fluctuations of speckles can be quantified using the electric field autocorrelation function g₁(τ). Typically g₁(τ) is difficult to measure and the intensity autocorrelation function g₂(τ) is recorded. The field and intensity autocorrelation functions are related through the Siegert relation,

g₂(τ)=1+β|g₁(τ)², Equation 2

where β is a normalization factor which accounts for speckle averaging due to mismatch of speckle size and detector size, polarization and coherence effects. In prior art, it was assumed that β=1 and Equation 2 was used, along with the fact that the recorded intensity is integrated over the exposure duration, to derive the first speckle model,

$$K ( T , {τ}_{c} ) = {( \frac{1 - {e}^{- 2 x}}{2 x} )}^{1 / 2} , \qquad Equation 3$$

where x=T/τ_{c}, T is the exposure duration of the camera and τ_{c} is the correlation time. Equation 3 has been widely used to determine relative blood flow changes for LSCI measurements.

Recently, it has been shown that Equation 3 did not account for speckle averaging effects. Arguing that β should not be ignored and also using triangular weighting of the autocorrelation function, a more rigorous model relating speckle contrast to τ_{c} was developed,

$$K ( T , {τ}_{c} ) = {( β \frac{{e}^{- 2 x} - 1 + 2 x}{2 {x}^{2}} )}^{1 / 2} . \qquad Equation 4$$

One disadvantage of these prior models is that they breakdown in the presence of statically scattered light. This is primarily because these models rely on the Siegert relation (Equation 2) which assumes that the speckles follow Gaussian statistics in time. However, in the presence of static scatterers, the fluctuations of the scattered field remain Gaussian but the intensity acquires an extra static contribution causing the recorded intensity to deviate from Gaussian statistics, and hence the Siegert relation (Equation 2) cannot be applied. This can be corrected by modeling the scattered field as

Eₕ(t)=E(t)+Eₛe^{iω}^{₀}^{lt}, Equation 5

where E(t) is the Gaussian fluctuation, Eₛ is the static field amplitude and ω₀ is the source frequency. The Siegert relation can now be modified as,

$$\begin{matrix}{g}_{2}^{h} ( τ ) = 1 + \frac{β}{{( {I}_{f} + {I}_{s} )}^{2}} [ {I}_{f}^{2} \vert {{g}_{1} ( τ )}^{2} \vert + 2 {I}_{f} {I}_{s} \vert {g}_{1} ( τ ) \vert ] \\ = 1 + A β \vert {{g}_{1} ( τ )}^{2} \vert + B β \vert {g}_{1} ( τ ) \vert ,\end{matrix} \\ where A = \frac{{I}_{f}^{2}}{{( {I}_{f} + {I}_{s} )}^{2}} and B = \frac{2 {I}_{f} {I}_{s}}{{( {I}_{f} + {I}_{s} )}^{2}} , {I}_{s} = {E}_{s} {E}_{S}^{*} \qquad Equation 6$$

represent contribution from the static scattered light, and I_{f}= [symbol] EE* [symbol] represent contribution from the dynamically scattered light.

This updated Siegert relation can be used to derive the relation between speckle variance and correlation time as with the other models. Following the approach of Bandyopadhyay et. al. the second moment of intensity can be written using the modified Siegert relation as

$$\begin{matrix}〈 {I}^{2} 〉 T ≡ {〈 {\int }_{0}^{T} {\int }_{0}^{T} {I}_{i} ( {t}^{′} ) {I}_{i} ( {t}^{″} ) d {t}^{′} d {t}^{″} / {T}^{2} 〉}_{i} \\ = {〈 I 〉}^{2} {\int }_{0}^{T} {\int }_{0}^{T} [ \begin{matrix}1 + A {β ( {g}_{1} ( {t}^{′} - {t}^{″} ) )}^{2} + \\ B β {g}_{1} ( {t}^{′} - {t}^{″} )\end{matrix} ] \\ d {t}^{′} d {t}^{″} / {T}^{2} .\end{matrix} \qquad Equation 7$$

The reduced second moment of intensity or the variance is hence

v₂(T)≡∫₀^{T} ∫₀^{T} [Aβ(g₁(t′−t″))²+Bβg₁(t′−t″)]dt′dt″/T². Equation 8

Since g₁(t) is an even function, the double integral simplifies to

$${v}_{2} ( T ) = A β {\int }_{0}^{T} 2 {( 1 - \frac{t}{T} ) [ {g}_{1} ( t ) ]}^{2} \frac{d t}{T} + B β {\int }_{0}^{T} 2 ( 1 - \frac{t}{T} ) [ {g}_{1} ( t ) ] \frac{d t}{T} \qquad Equation 9$$

Equation 9 represents a new speckle visibility expression that accounts for the varying proportions of light scattered from static and dynamic scatterers. Assuming that the velocities of the scatterers have a Lorentzian distribution, which gives g₁(t)=e^{-t/}^{^{τ}}^{c}, and recognizing that the square root of the variance is the speckle contrast, Equation 9 can be simplified to:

$$K ( T , {τ}_{c} ) = {\{ β {ρ}^{2} \frac{{e}^{- 2 x} - 1 + 2 x}{2 {x}^{2}} + 4 β ρ ( 1 - ρ ) \frac{{e}^{- x} - 1 + x}{{x}^{2}} \}}^{1 / 2} , \\ where x = \frac{T}{{τ}_{c}} , ρ = \frac{{I}_{f}}{( {I}_{f} + {I}_{s} )} \qquad Equation 10$$

is the fraction of total light that is dynamically scattered, β is a normalization factor to account for speckle averaging effects, T is the camera exposure duration and τ_{c} is the correlation time of the speckles.

When there are no static scatterers present, ρ→1 and Equation 10 simplifies to Equation 4. However Equation 10 is incomplete since in the limit that only static scatterers are present (ρ→0), it does not reduce to a constant speckle contrast value as one would expect for spatial speckle contrast. This can be explained by recognizing that K in Equation 10 refers to the temporal (temporally sampled) speckle contrast. The initial definition of K (Equation 1) was based on spatial sampling of speckles. Traditionally, in LSCI, speckle contrast has been estimated through spatial sampling by assuming ergodicity to replace temporal sampling of speckles with an ensemble sampling. In the presence of static scatterers this assumption is no longer valid. It is preferred to use spatial (ensemble sampled) speckle contrast because it helps retain the temporal resolution of LSCI. In order for the current theory to be used with spatial (ensemble sampled) speckle contrast, a constant term is added to the speckle visibility expression (Equation 9). This constant is referred to as nonergodic variance (vₙₑ). It is assumed that this is constant in time.

The speckle pattern obtained from a completely static sample does not fluctuate. Hence the variance of the speckle signal over time is zero as predicted by Equation 10. However the spatial (or ensemble) speckle contrast is a nonzero constant due to spatial averaging of the random interference pattern produced. This nonzero constant (vₙₑ) is primarily determined by the sample, illumination and imaging geometries. Since the speckle contrast is normalized to the integrated intensity, vₙₑ does not depend on the integrated intensity. These factors are clearly independent of the exposure duration of the camera, and hence the assumption is valid. The addition of vₙₑ allows the continued use of spatial (or ensemble) speckle contrast in the presence of static scatterers. This addition of the nonergodic variance is a significant improvement over existing models.

An additional factor that has been previously neglected is experimental noise which can have a significant impact on measured speckle contrast. Experimental noise can be broadly categorized into shot noise and camera noise. Shot noise is the largest contributor of noise, and it is primarily determined by the signal level at the pixels. This can be held independent of exposure duration, by equalizing the intensity of the image across different exposure durations. Camera noise includes readout noise, QTH noise, Johnson noise, etc. It can also be made independent of exposure by holding the camera exposure duration constant. The present disclosure provides a MESI system that holds camera exposure duration constant, yet obtains multi-exposure speckle images by pulsing the laser, while maintaining the same intensity over all exposure durations. Hence the experimental noise will add an additional constant spatial variance, vₙₒᵢₛₑ.

In the light of these arguments, Equation 10 can be rewritten as:

$$K ( T , {τ}_{c} ) = {\{ \begin{matrix}β {ρ}^{2} \frac{{e}^{- 2 x} - 1 + 2 x}{2 {x}^{2}} + \\ 4 β ρ ( 1 - ρ ) \frac{{e}^{- x} - 1 + x}{{x}^{2}} + {v}_{ne} + {v}_{noise}\end{matrix} \}}^{1 / 2} , \\ where x = \frac{T}{{τ}_{c}} , ρ = \frac{{I}_{f}}{( {I}_{f} + {I}_{s} )} \qquad Equation 11$$

is the fraction of total light that is dynamically scattered, β is a normalization factor to account for speckle averaging effects, T is the camera exposure duration, τ_{c} is the correlation time of the speckles, vₙₒᵢₛₑ is the constant variance due to experimental noise and vₙₑ is the constant variance due to nonergodic light.

Equation 11 is a rigorous and practical speckle model that accounts for the presence of static scattered light, experimental noise and nonergodic variance due to the ensemble averaging. While vₙₑ and vₙₒᵢₛₑ make the model more complete, they do not add any new information about the dynamics of the system, all of which is held in τ_{c}. Hence vₙₑ and vₙₒᵢₛₑ can be viewed as experimental variables/artifacts. In the present disclosure, vₙₑ and vₙₒᵢₛₑ may be combined as a single static spatial variance vₛ, where vₛ=vₙₑ+vₙₒᵢₛₑ.

Accordingly, the speckle model of the present disclosure (Equation 11) accounts for the presence of light scattered from static particles. The model of the present disclosure applies the theory of time integrated speckle to static scattered light. The model of the present disclosure also takes into account the assumption that ergodicity breaks down in the presence of static scatterers and thus proposes a solution to account for nonergodic light. Furthermore, the speckle model of the present disclosure provides a model that accounts for experimental noise. The influence of noise and nonergodic light have been neglected in most previous studies.

The methods of the present disclosure may be implemented in software to run on one or more computers, where each computer includes one or more processors, a memory, and may include further data storage, one or more input devices, one or more output devices,, and one or more networking devices. The software includes executable instructions stored on a tangible medium.

It should be noted that the speckle model of the present disclosure generally works when the speckle signal from dynamically scattered photons is strong enough to be detected in the presence of the static background signal. If the fraction of dynamically scattered photons is too small compared to statically scattered photons, the dynamic speckle signal would be insignificant and estimates of τ_{c} breakdown. For practical applications, a simple single exposure LSCI image or visual inspection can qualitatively verify if there is sufficient speckle visibility due to dynamically scattered photons and subsequently the model of the present disclosure can be used to obtain consistent estimates of correlation times.

B. Multi-Exposure Speckle Imaging System

In addition to the speckle model presented above, the present disclosure also provides a MESI system. In some embodiments, a MESI system of the present disclosure is able to acquire images that will obtain correlation time information. Additionally, in some embodiments, a MESI system of the present disclosure is able to vary the exposure duration, maintain a constant intensity over a wide range of exposures and ensure that the noise variance is constant.

In one embodiment, a MESI system of the present disclosure generally comprises a laser light source; a light modulator; and a detector for the measurement of reflected light comprising at least one camera, at least one magnification objective, and at least one microprocessor or data acquisition unit. Examples of suitable light modulators may include, but are not limited to, an acousto-optic modulator, an electro-optic modulator, or a spatial light modulator.

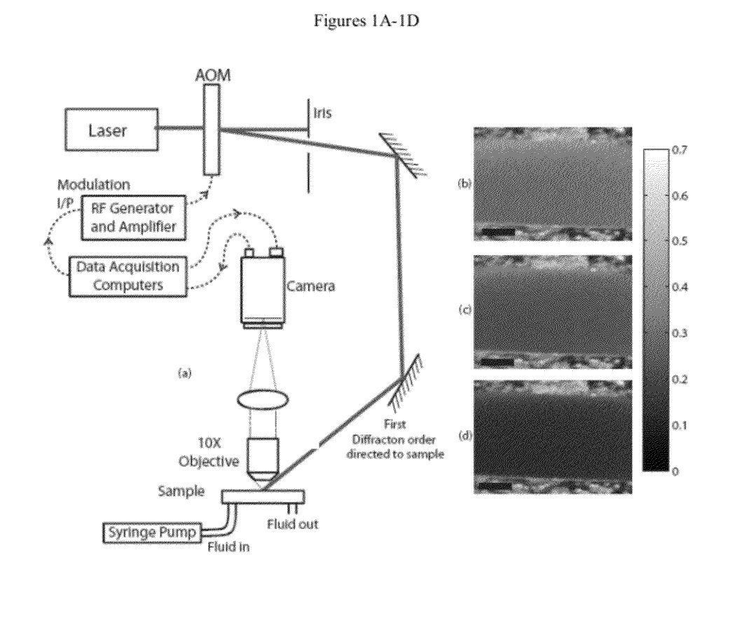

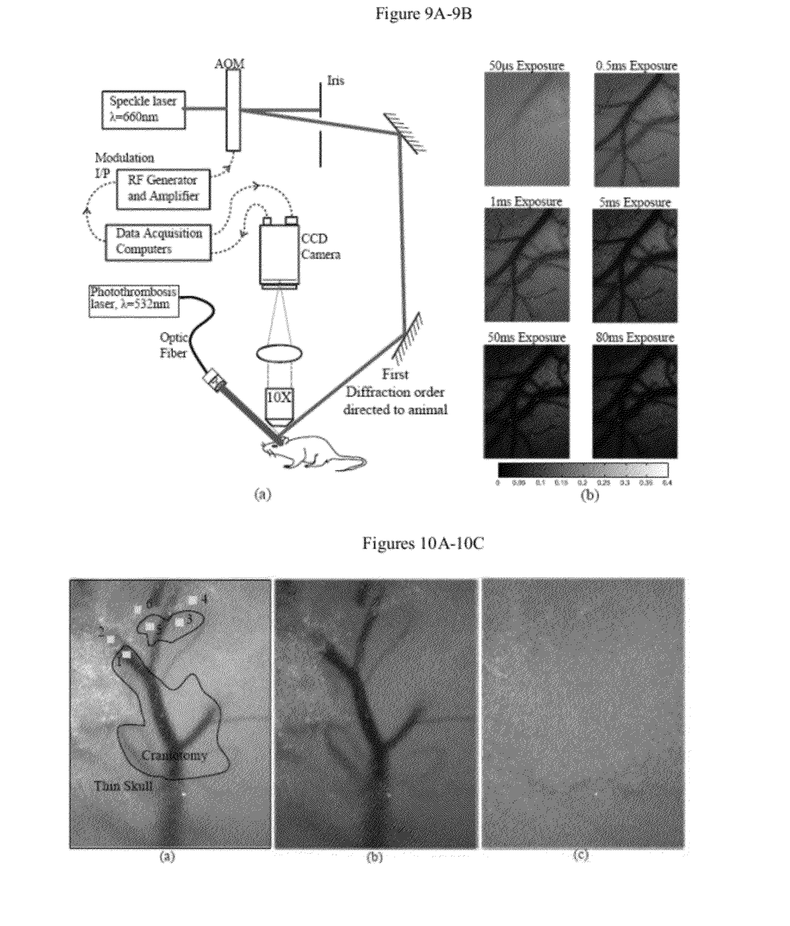

A MESI system of the present disclosure may also comprise additional electronic and mechanical components such as a gated laser diode, a digitizer, a motion controller, a stepper motor, a trigger, a delay switch, and/or a display monitor. One of ordinary skill in the art, with the benefit of this disclosure, will recognize additional electronic and mechanical components that may be suitable for use in the methods of the present invention. Furthermore, a MESI system of the present disclosure may also be used in conjunction with custom-made software. An example of an embodiment of a MESI system is depicted in FIG. 1A and FIG. 9A.

The need for high-resolution blood flow imaging spans many applications, tissue types, and diseases. Accordingly, the MESI systems of the present disclosure may be used in a variety of applications, including, but not limited to, blood imaging applications in tissues such as the retina, skin, and brain. In another embodiment, the MESI systems of the present disclosure may be used during surgery.

### EXAMPLE 1

The examples provided herein utilize a tissue phantom to show that the speckle model of the present disclosure, used in conjunction with a MESI system of the present disclosure, can predict correlation times consistently in the presence of static speckles.

In order to test the model experimentally, flow measurements were performed on microfluidic flow phantoms. To do this, the exposure duration of speckle measurements had to be changed, while ensuring that certain conditions were satisfied. To obtain speckle images at multiple exposure durations, the actual camera exposure duration was fixed and a laser diode was gated during each exposure to effectively vary the speckle exposure duration T as in Yuan et. al. This approach ensures that the camera noise variance and the average image intensity is constant. Directly pulsing the laser limited the range of exposure durations that can be achieved. The lasing threshold of the laser diode dictated the minimum intensity and hence the maximum exposure duration that could be recorded. Consequently, the minimum exposure duration was limited by the dynamic range of the instruments. To overcome this limitation, the laser was pulsed through an acousto-optic modulator (AOM). By modulating the amplitude of the radio-frequency wave fed to the AOM, the intensity of the first diffraction order could be varied, enabling control over both the integrated intensity and the effective exposure duration.

FIG. 1 provides a schematic representation of the MESI system used in this example. A diode laser beam (Hitachi HL6535MG; λ=658 nm, 80 mW Thorlabs, Newton, N.J., USA) was directed to an acousto-optic modulator (AOM) (IntraAction Corp., BellWood, Ill., USA). The AOM was driven by signals generated from an RF AOM Modulator driver (IntraAction Corp., BellWood, Ill., USA) and the first diffraction order was directed towards the sample. The sample was imaged using a 10×∞ corrected objective (Thorlabs, Newton, N.J., USA) and a 150 mm tube lens (Thorlabs, Newton, N.J., USA). Images were acquired using a camera (Basler 602f; Basler Vision Technologies, Germany). Software was written to control the timing of the AOM pulsing and synchronize it with image acquisition.

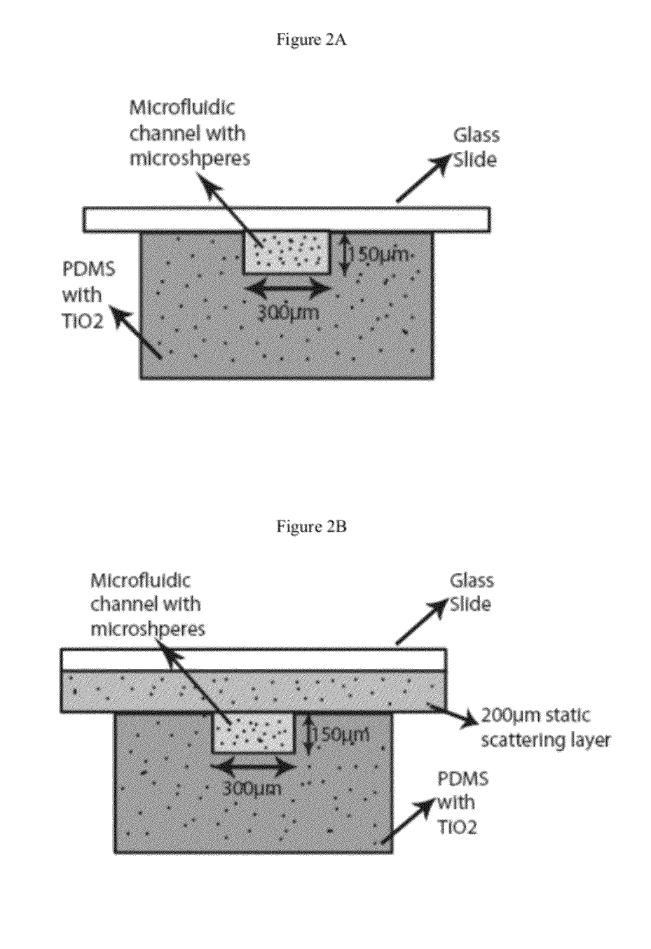

A microfluidic device was used as a flow phantom in this example. A microfluidic device as a flow phantom has the advantage of being realistic and cost effective, providing flexibility in design, large shelf life and robust operation. A microfluidic device without a static scattering layer (FIG. 2A) and with a static scattering layer (FIG. 2B) were prepared. The channels were rectangular in cross section (300 μm wide×150 μm deep). The microfluidic device was fabricated in poly dimethyl siloxane (PDMS) using the rapid prototyping technique disclosed in J. Anderson, D. Chiu, R. Jackman, O. Cherniayskaya, J. McDonald, H. Wu, S. Whitesides, and G. Whitesides, “Fabrication of Topologically Complex Three-Dimensional Microfluidic Systems in PDMS by Rapid Prototyping,” Science 261, 895 (1993). Titanium dioxide (TiO₂) was added to the PDMS (1.8 mg of TiO₂ per gram of PDMS) to give the sample a scattering background to mimic tissue optical properties. The prepared samples were bonded on a glass slide to seal the channels as shown in FIGS. 2A and 2B. The sample was connected to a mechanical syringe pump (World Precision Instruments, Saratosa, Fla., USA) through silicone tubes, and a suspension (μ′ₛ=250 cm⁻¹) of 1 μm diameter polystyrene beads (Duke Scientific Corp., Palo Alto, Calif., USA) (“microspheres”) was pumped through the channels.

For the static scattering experiments, a 200 μm layer of PDMS with different concentrations of TiO₂ (0.9 mg and 1.8 mg of TiO₂ per gram of PDMS corresponding to (μ′ₛ=4 cm⁻¹) and (μ′ₛ=8 cm⁻¹ respectively) was sandwiched between the channels and the glass slide, to simulate a superficial layer of static scattering such as a thinned skull (FIG. 2B). The reduced scattering coefficients of the 200 μm static scattering layer were estimated using an approximate collimated transmission measurement through a thin section of the sample. FIGS. 2A and 2B show a schematic of the cross-section of the devices.

The experimental setup (FIG. 1) was used in conjunction with the exposure modulation technique to perform controlled experiments on the microfluidic samples. The microfluidic sample without the static scattering layer (FIG. 2A) was used to test the accuracy of the MESI system and the speckle model. As detailed earlier, the suspension of microspheres was pumped through the sample using the syringe pump at different speeds from 0 mm/sec (Brownian motion) to 10 mm/sec in 1 mm/sec increments. 30 speckle contrast images were calculated and averaged for each exposure from the raw speckle images. The average speckle contrast in a region within the channel was calculated.

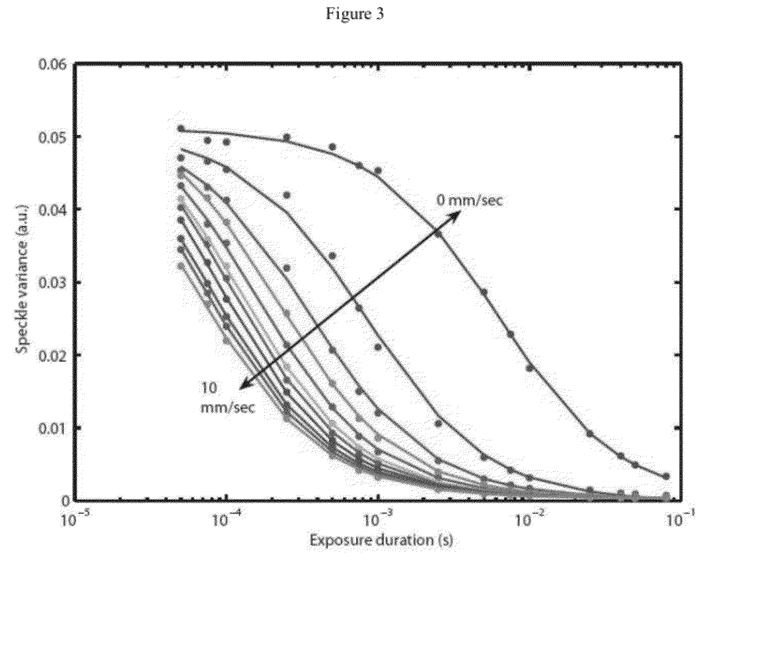

In this fully dynamic case, the static spatial variance vₛ is very small. vₛ would be dominated by the experimental noise vₙₒᵢₛₑ as the ergodicity assumption would be valid and vₙₑ≈0. β is one of the unknown quantities in Equation 11 describing speckle contrast. Theoretically, β is a constant that depends only on experimental conditions. An attempt to estimate β using a reflectance standard would yield inaccurate results due to the presence of the static spatial variance vₛ. Here the ergodicity assumption would breakdown, and vₙₑ would be significant. It would not be possible to separate the contributions of speckle contrast from β, vₙₑ and vₙₒᵢₛₑ. Instead, the value of β was estimated, by performing an initial fit of the multi-exposure data to Equation 4 with the addition of vₛ, while having β, τ_{c} and vₛ as the fitting variables. The speckle contrast data was then fit to Equation 11 using the estimated value of β and the results are shown in FIG. 3. Holding β constant ensures that the fitting procedure is physically appropriate and makes the nonlinear optimization process less constrained and computationally less intensive. FIG. 3 clearly shows that the model fits the experimental data very well (mean sum squared error: 2.4×10⁻⁶). The correlation time of speckles was estimated by having τ_{c} as a fitting parameter. The standard error of correlation time estimates was found using bootstrap resampling. Correlation times varied from 3.361±0.17 ms for Brownian motion to 38.4±1.44 μs for 10 mm/sec. The average percentage error in estimates of correlation times was 3.37%, with a minimum of 1.99% for 3 mm/sec and a maximum of 5.2% for Brownian motion. Other fitting parameters were vₛ, the static spatial variance and ρ, the fraction of dynamically scattered light. A priori knowledge of ρ was not required to obtain τ_{c} estimates. Hence this technique can be applied to cases where the thickness of the skull is unknown and/or variable.

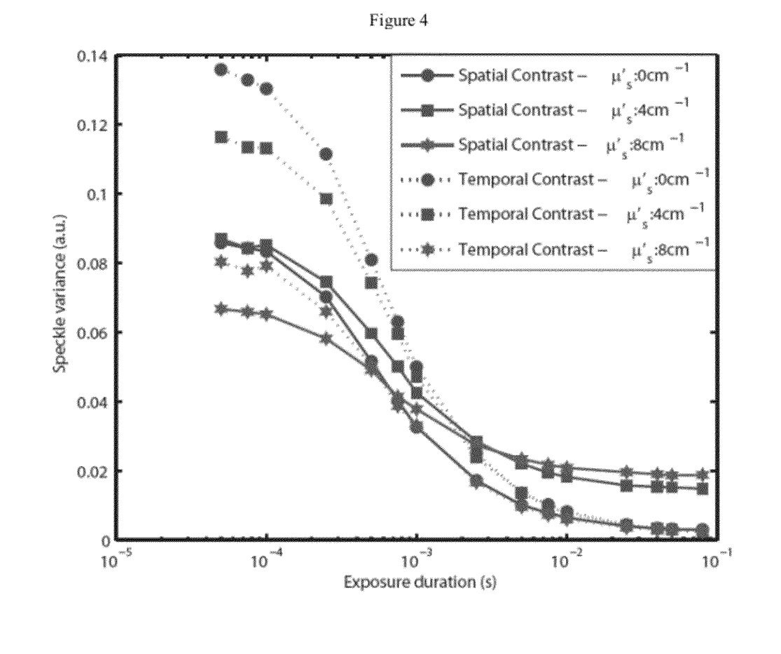

In order to verify the arguments on nonergodicity, the speckle contrast obtained using spatial analysis and temporal analysis was compared. Spatial speckle contrast was estimated by using Equation 1 and the procedure detailed earlier, while temporal speckle contrast was estimated by calculating the ratio of the standard deviation to mean of pixel intensities over different frames at the same exposure duration. Multi-exposure speckle contrast measurements were performed on the microfluidic devices with different levels of static scattering in the static scattering upper layer (FIG. 2A: μ′ₛ=0 cm⁻¹ and FIG. 2B: μ′ₛ=4 cm⁻¹ and μ′ₛ=8 cm⁻¹). A suspension (μₛ=250 cm⁻¹) of 1 μm diameter polystyrene beads was pumped through the channels at 2 mm/sec. The experimentally obtained temporal contrast (temporal sampling) and spatial contrast (ensemble sampling) curves for each static scattering case is shown in FIG. 4.

From FIG. 4, it can be seen that the temporal contrast curves (dotted lines) do not possess a significant constant variance since the variance approaches zero at long exposure durations. The small offset that was observed was likely due to vₙₒᵢₛₑ which remains constant even in the presence of static scattering and does not change as the amount of static scattering increases. However, the spatial (ensemble sampled) contrast curves (solid lines) show a clear offset at large exposure durations when static scatterers were present. This offset increases with an increase in static scattering. Again, when no static scatterers were present, the spatial (ensemble sampled) contrast curve does not possess this offset. The speckle variance curves show that the nonergodic variance vₙₑ is absent in all three temporally sampled curves and in the completely dynamic spatially (ensemble) sampled curve. vₙₑ is significant in the cases with a static scattered layer, when the data is analyzed by spatial (ensemble) sampling. This provides evidence in favor of the argument that the increase in variance at large exposure durations is due to vₙₑ, the nonergodic variance. For the same static scattering level, the variance obtained by temporal sampling is greater than the variance obtained by spatial sampling. This could be due to different β. The objective was not to compare temporal speckle contrast with spatial speckle contrast, but to utilize the two curves to provide evidence in favor of the model.

One of the significant improvements that the speckle model of the present disclosure provides is its ability to estimate correlation times consistently in the presence of static scatterers. The flow measurements as detailed earlier were repeated, at speeds 0 mm/sec to 10 mm/sec in 2 mm/sec increments. Measurements on the sample with no static scattering layer (FIG. 2A) served as base (or ‘true’) estimates of correlation times. FIG. 5 shows the results of this analysis at two different speeds. The addition of the static scattering layer drastically changed the shape of the curve. For a given speed, the decrease in variance at the low exposures was due to the relative weighting of the two exponential decays in Equation 11 which was consistent with results obtained with DLS measurements. The increase in variance at the larger exposure durations was due to the addition of the nonergodic variance vₙₑ. The speckle model of the present disclosure fit well to the data points. Also, the ρ values decreased with the addition of static scattering, implying a reduction in the fraction of total light that was dynamically scattered. It is important to note that for a given exposure duration and speed, the measured speckle contrast values were different in the presence of static scattered light when compared to the speckle contrast values obtained in the absence of static scattered light. Hence accurate τ_{c} estimates cannot be obtained with measurements from a single exposure duration without an accurate model and a priori knowledge of the constants ρ, β and vₛ. These constants are typically difficult to estimate. By using the multi-exposure data and the speckle model of the present disclosure, this problem was overcome and τ_{c} was reproduced consistently.

To quantify the effects of the static scattering layer on the consistency of the τ_{c} estimates, the deviations in τ_{c} were estimated for each speed as the amount of static scatterer was varied. For each speed, the variation in the estimated correlation times over the three scattering cases (FIG. 2A: μ′ₛ=0 cm⁻¹ and FIG. 2B: μ′ₛ=4 cm⁻¹ and μ′ₛ=8 cm⁻¹) was estimated by calculating the standard deviation of the correlation time estimates.

$$\% Deviation in {τ}_{c} = \frac{Standard deviation in {τ}_{c}}{{τ}_{c} in the absence of static scatters} \times 100$$

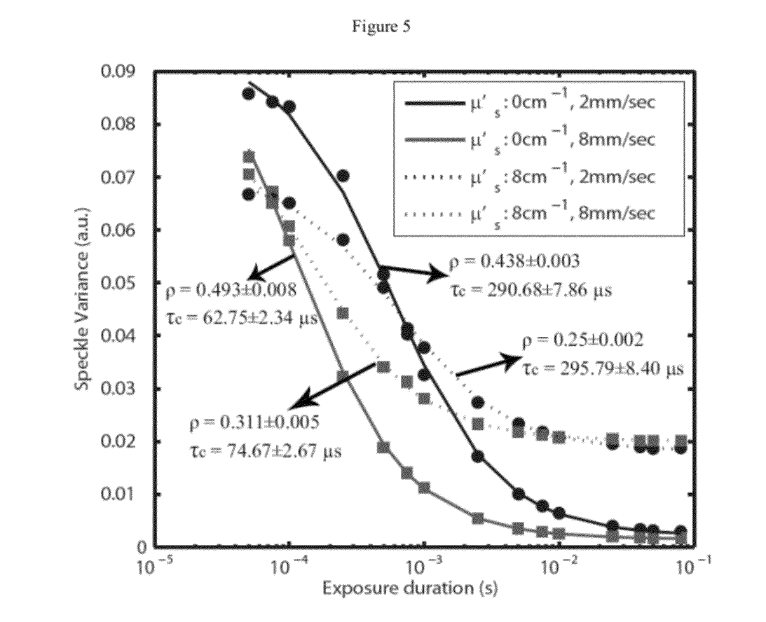

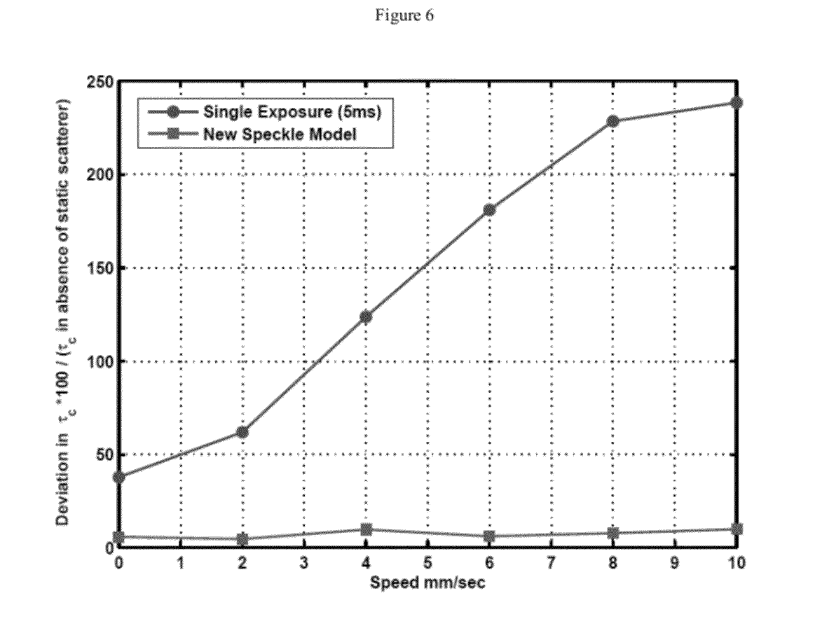

This deviation was normalized to the base (or ‘true’) correlation time estimates. Single exposure estimates of correlation time was obtained using Equation 3. Equation 3 was used in estimating the correlation time because of its widespread use in most speckle imaging techniques to estimate relative flow changes, and was hence most appropriate for this comparison. The correlation time was estimated from a lookup table. A lookup table which relates speckle contrast values to correlation times was generated using Equation 3 for the given exposure time. The correlation time was then estimated through interpolation from the lookup table for the appropriate speckle contrast value. For an appropriate comparison, β was prefixed to Equation 3, and same value of β was used for both the single exposure and MESI estimates. The results for the speckle model of the present disclosure and the single exposure case are plotted in FIG. 6.

FIG. 6 shows that the single exposure estimates are not suited for speckle contrast measurements in the presence of static scatterers. The error in the correlation time estimates is high and increases drastically with speed. The speckle model of the present disclosure performed very well, with deviation in correlation times being less than 10% for all speeds. τ_{c} estimates with the speckle model of the present disclosure have extremely low deviation. This shows that the speckle model of the present disclosure can estimate correlation times consistently even in the presence of static scattering.

The lack of quantitative accuracy of correlation time measures using LSCI can be attributed to several factors including inaccurate estimates of β and neglect of noise contributions and nonergodicity effects. The absence of the noise term in traditional speckle measurements can also lead to incorrect speckle contrast values for a given correlation time and exposure duration. A MESI system of the present disclosure reduces this experimental variability in measurements. Since images are obtained at different exposure durations, the integrated autocorrelation function curve can be experimentally measured, and a speckle model can be fit to it to obtain unknown parameters, which include the characteristic decay time or correlation time τ_{c}, experimental noise and in the speckle model of the present disclosure, ρ, the fraction of dynamically scattered light. A MESI system of the present disclosure also removes the dependence of vₙₒᵢₛₑ on exposure duration. The speckle model of the present disclosure and the τ_{c} estimation procedure allows for determination of noise with a constant variance. Without these improvements it would be very difficult to separate the variance due to speckle decorrelation and the lumped variance due to noise and nonergodicity effects.

### EXAMPLE 2

Another experiment was conducted to test whether the τ_{c} estimates obtained using a MESI system of the present disclosure were more accurate than traditional single exposure LSCI measures by comparing the respective estimates of the relative correlation time measures. Correlation time estimates from traditional single exposure measures were obtained using the procedure detailed earlier. Relative correlation time measures were defined as:

$$relative {τ}_{c} = \frac{{t}_{co}}{{τ}_{c}} , \qquad ( 12 )$$

where τ_{co} is the correlation time at baseline speed and τ_{c} is the correlation time at a given speed. Correlation time estimates were obtained from the fits performed in FIG. 3, on multi-exposure speckle contrast data obtained with measurements made on the fully dynamic sample (FIG. 2A). The τ_{c} estimates obtained with the MESI instrument were compared with traditional single exposure estimates of ρ_{c} at 1 ms and 5 ms exposures for their efficiency in predicting relative flows. Ideally, relative correlation measures would be linear with relative speed. Relative correlation times were obtained for a baseline flow of 2 mm/sec.

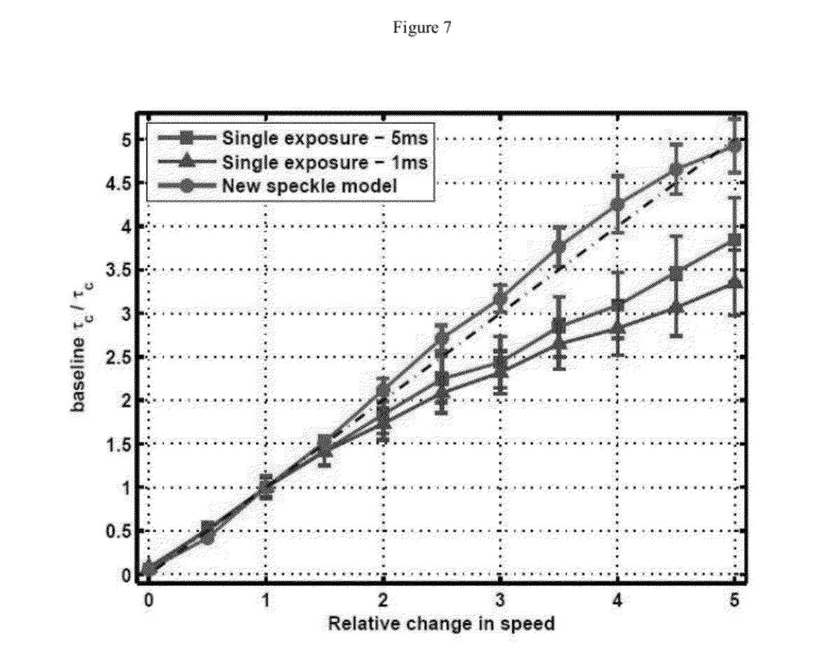

FIG. 7 shows that the speckle model of the present disclosure used in conjunction with a MESI system of the present disclosure maintains linearity of relative correlation measures over a long range. Single exposure estimates of relative correlation measures are linear for small changes in flows, but the linearity breaks down for larger changes. A MESI system and the speckle model of the present disclosure address this underestimation of large changes in flow by traditional LSCI measurements. This comparison is significant, because relative correlation time measurements are widely used in many dynamic blood flow measurements. Traditional single exposure LSCI measures underestimate relative flows for large changes in flow. This example shows that a MESI system of the present disclosure and the speckle model of the present disclosure can provide more accurate measures of relative flow.

FIG. 7 also shows that even in a case where there is no obvious static scatterer like a thinned skull, there appears to be some contributions due to static scatterers, in this case possibly from the bottom of the channel in FIG. 2A. While the fraction of static scatterers is not too significant, it appears to affect the linearity of the curve, and a MESI system of the present disclosure with the speckle model of the present disclosure can eliminate this error.

### EXAMPLE 3

As shown earlier, the presence of static scatterers significantly alters the shape of the integrated autocorrelation function curve in FIG. 5, for different speeds. Also, it was previously shown that the speckle model of the present disclosure fits well to the experimentally determined speckle variance curve (FIG. 5) and that the speckle model provides consistent estimates of τ_{c} even in the presence of static scatterers (FIG. 6). This example tested whether the correlation time estimates obtained with a MESI system of the present disclosure and the speckle model maintained linearity for relative flow measurements (as in FIG. 7) in the presence of static scatterers.

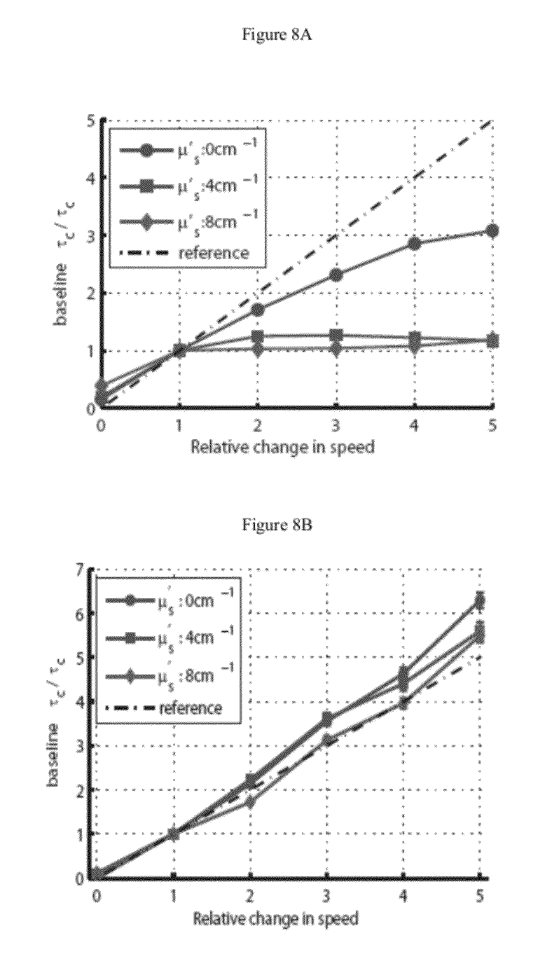

Relative correlation time measures were obtained as detailed earlier (Equation 12) using 2 mm/sec as the baseline measure. The speckle model of the present disclosure and traditional single exposure measurements (5 ms) were evaluated, and the results are shown in FIG. 8. FIG. 8 shows again why traditional single exposure methods are not suited for flow measurements when static scatterers are present. The linearity of relative correlation time measurements with single exposure measurements breaks down in the presence of static scatterers (FIG. 8A) while the speckle model of the present disclosure maintains the linearity of relative correlation time measures even in the presence of static scatterers (FIG. 8B). This again reinforces the fact that a MESI system and the speckle model of the present disclosure can predict consistent correlation times in the presence of static scatterers.

### EXAMPLE 4

Materials and Methods

A Multi Exposure Speckle Imaging (MESI) instrument according to one embodiment is shown in FIG. 9A. Speckle images at different camera exposure durations were acquired by triggering a camera (Basler 602f, Basler Vision Technologies, Germany) and simultaneously gating a laser diode (λ=660 nm, 95 mW, Micro laser Systems Inc., Garden Grove, Calif., USA) with an acousto-optic modulator to equalize the energy of each laser pulse. The first diffraction order was directed towards the animal, and the backscattered light was collected by a microscope objective (10×) and imaged onto the camera. By appropriately controlling the acousto-optic modulator, the intensity of light in the first diffraction order and hence the average intensity recorded by camera was maintained a constant over different exposure durations.

Laser speckle images were collected at 15 different exposure durations from 50 μs to 80 ms, and the entire setup was controlled by custom software. Spatial speckle contrast images was computed using a window size of N=7. FIG. 9B shows some speckle contrast images of the mouse cortex at different camera exposure durations. These images span almost 3 orders of magnitude of exposure duration which is possible with an inexpensive camera using the MESI approach.

In one embodiment, a method of the present disclosure involves the use of a MESI instrument (FIG. 9A) in conjunction with a mathematical model, represented by Equation 11, that relates the speckle contrast to the camera exposure duration, T and the decay time of the speckle autocorrelation function, τc. This model is designed to account for the heterodyne mixing of light scattered from static and moving particles, as well as the contributions of nonergodic light and experimental noise to speckle variance.

Animal Preparation

The methods of the present disclosure were used to image cerebral blood flow changes that occur during ischemic stroke in mice. Mice (CD-1; male, 25-30 g, n=5) were used for these experiments. All experimental procedures were approved by the Animal Care and Use Committee at the University of Texas at Austin. The animals were anesthetized by inhalation of 2-3% isoflurane in oxygen through a nose cone. Body temperature was maintained at 37 C using a feedback controlled heating plate (ATC100, World Percision Instruments, Sarasota, Fla., USA) during the experiment. The animals were fixed in a stereotaxic frame (Kopf Instruments, Tujunga, Calif., USA) and a ˜3 mm×3 mm portion of the skull was exposed by thinning it down using a dental burr burr (IdealTM Micro-Drill, Fine Science tools, Foster City, Calif., USA). Further, part of this thinned skull was removed to create a partial craniotomy (shown in FIG. 10A). Care was taken to ensure that the boundary between the thin skull and the craniotomy was over a vessel and that the boundary was away from major branches. This ensured that one can expect the same blood flow changes across the boundary. The partial craniotomy was completed by building a well around the region using dental cement and filling it with mineral oil. The surgery was supplemented with subcutaneous injections of Atropine (0.04 mg/kg) every hour to prevent respiratory difficulties and intraparetonial injections of dextrose-saline (2 ml/kg/h of 5% w/v) for hydration.

Ischemic Stroke Using Photothrombosis

To induce an ischemic stroke, the middle cerebral artery (MCA) was occluded using photothrombosis. During animal preparation, the temporalis muscle in the same hemisphere of the craniotomy was carefully resected from the temporal bone. The temporal bone was then thinned using the dental burr till it was transparent and the MCA was visible. A laser beam (λ=532 nm, Spectra Physics, Santa Clara, Calif., USA) was directed towards the MCA through an optical fiber. Typical laser power delivered to the animal during the experiment was ˜0.5-0.75 W. During the experiment, a 1 ml bolus intraparetonial injection of a photosensitive thrombotic agent Rose Bengal (15 mg/kg) was administered to the animal. The laser light interacts with the Rose Bengal to cause thrombosis in the MCA resulting in occlusion. FIGS. 10B and 10C show LSCI images (at 5 ms exposure) before and after the stroke was induced. Occluding the MCA created a severe stroke and reduced blood flow by almost 100% in the cortical regions downstream.

Imaging Paradigm

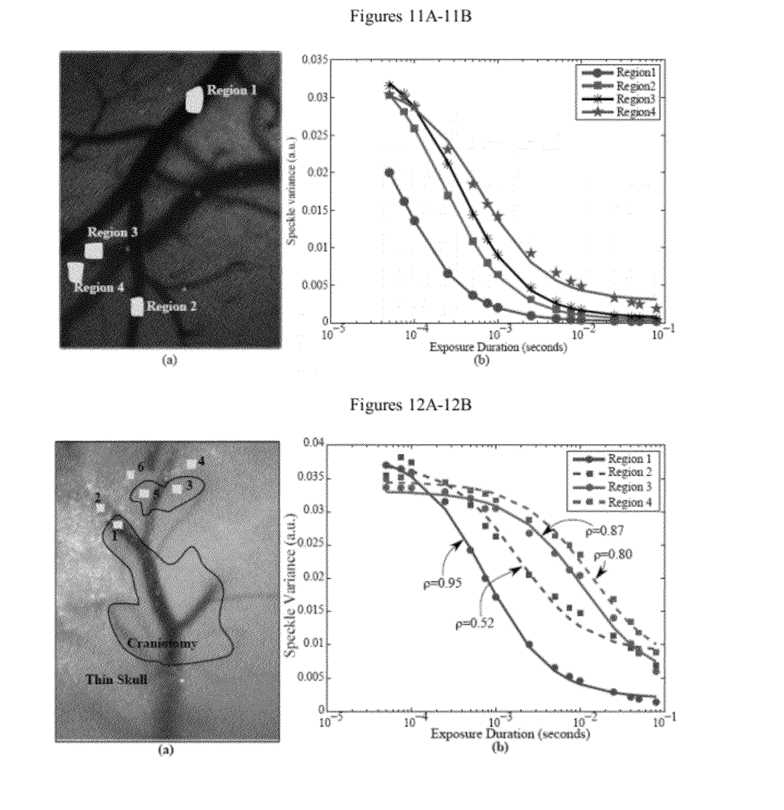

The experimental setup shown in FIG. 9A was used to acquire multi exposure speckle images before, during and after the stroke. Laser speckle images at 15 exposure durations ranging from 50 μs to 80 ms were used to compile one MESI frame. Typically, 3000 MESI frames were collected for each experiment. Each MESI frame took ˜1.5 seconds to acquire. The field of view of the cortex as measured by the MESI instrument was ˜800×500 μm. Specific regions of interest as shown in FIG. 11A were identified, and the average speckle contrast in these regions were computed for all MESI frames to produce the time integrated speckle contrast curves shown in FIG. 11B. Each curve was then fit to Equation 11 to estimate blood flow (τc).

Results

Estimating blood flow using methods of the present disclosure, FIG. 11 illustrates the first step in obtaining blood flow estimates. In this example, a MESI instrument (FIG. 9A) was used to obtain raw speckle images at multiple exposure durations of a mouse brain whose cortex had been exposed by performing a full craniotomy. After converting these raw images to speckle contrast images, specific regions of interest were identified (FIG. 11A), and the average speckle contrast in these regions were computed and plotted as a function of camera exposure duration (FIG. 11B). These experimentally measured time integrated speckle variance, K(T,τc) 2 curves were then fit to Equation 11 using the Trust-Region algorithm to obtain estimates for blood flow (through τc, the decay time of the speckle autocorrelation function). The curves correspond to different regions shown in FIG. 11A. From these curves, it can be observed that the variance decays with a lower τc value (and hence higher blood flow) in region 1 which is in the middle of a major vessel (a vein), when compared to region 4 which is in the parenchyma.

Imaging Blood Flow Changes Due to Ischemic Stroke

For stroke experiments, the partial craniotomy procedure was followed during animal preparation. A representative image of this model is shown in FIG. 12A. Regions 1, 3, and 5 are in the craniotomy, while regions 2, 4 and 6 are under the thin skull. MESI images were obtained and the blood flow was estimated using the procedures described in the previous section. FIG. 12B shows how the time integrated speckle variance curves are different for two regions across the thin skull boundary. The primary points of difference between the curves obtained from regions across the boundary are (a) an apparent change in the shape of the time integrated speckle variance curve over the thin skull due to variation in ρ (the fraction of light that is dynamically scattered), and (b) an increase in the variance at the longer exposure durations due to an increase in vₛ (the constant spatial variance that accounts for nonergodicity and experimental noise). This difference is more apparent in the regions on the vessel (regions 1 and 2) than it is in regions in the parenchyma (regions 3 and 4). With LSCI at a single exposure, regions 1 and 2 measure vastly different speckle contrast values even though the actual blood flow is likely identical. Under baseline conditions, the ratio of the correlation time in region 1 to the correlation time in region 2 was found to be 0.6238±0.0238 using the methods of the present disclosure, while this ratio was estimated to be 0.3771±0.0215 using the LSCI technique. While the ideal value for these ratios should be 1, these estimates suggest that the methods of the present disclosure predict τc values that are more consistent across the thin skull boundary. The ratio of the correlation time in region 3 to the correlation time in region 4 was found to be 0.883±0.055 using the methods of the present disclosure, while this ratio was estimated to be 0.889±0.019 using the LSCI technique. Both estimates of these ratios are similar over the parenchyma regions because the thickness of the thinned skull is nonuniform and was found to be thinner, as evidenced by higher values of ρ in region 4 compared to region 2.

Each stroke experiment was performed after waiting for about 30 minutes after surgical preparation. The first 10 minutes of the data was used as baseline measures to compute the relative blood flow change. The thrombosis inducing laser was kept on during the entire course of the experiment. Rose Bengal was injected 10 minutes after start of the experiment and data collection was continued for about an hour. Data acquisition was not stopped while the dye was being injected. Immediately after the completion of data acquisition, the animal (n=2) was sacrificed and 30 MESI frames (1 MESI frame consists of 15 exposure durations) were collected as a zero flow reference.

Since β is an experimental constant, its in vivo determination is important to obtain accurate flow measures. In addition to β, ρ and vₛ also have to be determined in vivo. However, we contend that changes in the physiology can change ρ and vₛ, and hence these parameters were not held fixed during the fitting process. First, β was estimated under baseline conditions for the regions in the craniotomy (regions 1, 3 and 5 shown in FIG. 12A), by using equation 11 and holding ρ=1. A statistical average of the estimated values of β were found for each region and this average value was used for the corresponding pair. For example, the value of β estimated from region 1, would be used for regions 1 and 2. The MESI curves from entire data set was then fit to Equation 11 using the estimated value of β, and holding it constant. Unknown parameters ρ, vₛ and the flow measure τc were estimated from this fitting process.

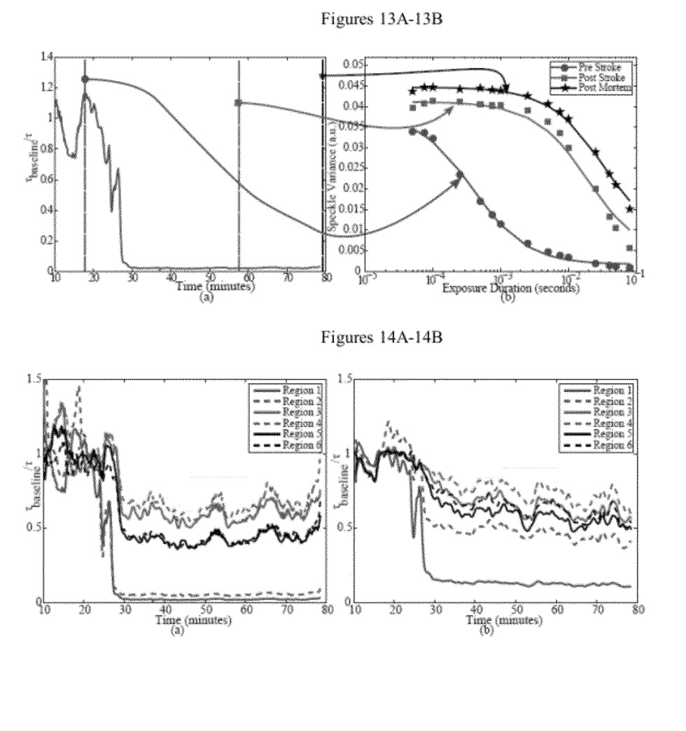

FIG. 13A shows the relative blood flow change as measured using the methods of the present disclosure in region 1, in the same animal as in FIG. 12. Since τc can be assumed to be inversely related to blood flow, relative blood flow may be defined as the ratio of τ baseline to τ measured. Here, τ baseline is the statistical average of the correlation time estimates during the first 10 minutes. From time t=10 min to t=30 min, the blood flow is seen to fluctuate. These fluctuations are due to the increase and decrease of blood flow while the clot is being formed in the MCA. For the MCA to be completely occluded, the photo thrombosis process has to create enough thrombus to occlude the vessel and its downstream branches. Since the MCA is a major artery, partially formed thrombus can be washed down by blood pressure. The partially formed clots break down and produce blood flow fluctuations. These fluctuations were observed in all animals before the stroke was formed. Once the thrombosis process is complete, the blood flow settles to a stable value. FIG. 13A shows that the relative blood flow drops to almost 0 after the clot is fully formed. The average percentage reduction in blood flow in the blood vessel, due to the ischemic stroke in all animals was estimated to be 97.3±2.09% using the methods of the present disclosure and 87.67±7.04% using the LSCI technique. The estimates of average percentage reduction in blood flow obtained using the methods of the present disclosure were found to be statistically greater than those obtained using the LSCI technique with a 5% significance level.

In FIG. 13B, three representative time integrated speckle variance curves estimated from region 1 (FIG. 12A) were shown as a function of camera exposure duration, illustrating the progression of the stroke in one representative animal. The first two curves are the time integrated speckle variance curves before and after ischemic stroke. The drastic change in the shape of the curve reinforces the observation that the change in blood flow is drastic, as previously noted in FIG. 10 and FIG. 13A. The shape of the curve after the stroke has been induced is indicative of Brownian motion. This trend was observed in all animals, and is comparable to similar measurements in literature. An experimental measurement of the time integrated speckle variance curve after the animal has been sacrificed (comparing the blue and black curves in FIG. 13B) further confirm these observations. In region 1 the average percentage reduction in blood flow due to death in all animals was estimated to be about 99% using the methods of the present disclosure and 92% using the LSCI technique. Since after death, the blood flow in the animals should be zero, it was concluded that the MESI technique has greater accuracy in predicting large flow decreases. This observation is consistent with previous measurements in phantoms discussed above.

While the post stroke and post mortem time integrated speckle variance curves are similar, the variances are different. The increase in measured speckle variance after the animal has been sacrificed is indicative of a further drop in blood flow. This drop is measured as a mild increase in τc. One of the reasons for the difference in speckle variance between the post stroke and the post mortem cases, is that in the post stroke case, the speckle contrast can still be affected by blood flow from deeper tissue regions (though not spatially resolved) which could possibly be unaffected by thrombosis. Additionally, the pulsation of the cortex in a live animal contributes to a reduction in variance. In the post mortem case, this pulsation is absent, and the blood flow is truly zero over the entire cortex. The only motion detected is due to limited (thermal induced) Brownian motion that can be associated with the dead cells. These factors coupled with physiological noise contribute to the difference in variance between the post mortem and the post stroke, cases. From these observations, we conclude that the magnitude of the blood flow reduction measured by methods of the present disclosure are accurate.

Imaging Blood Flow Changes Through the Thin Skull

FIG. 14 compares the relative blood flow measures as estimated by (A) the methods of the present disclosure and (B) LSCI technique at 5 ms exposure duration. 5 ms exposure duration was selected for comparison because it has been demonstrated to be sensitive to blood flow changes in vivo. Considering the first pair of regions across the thin skull boundary (regions 1 and 2 in FIG. 12A), the relative blood flow measures as estimated by the methods of the present disclosure (solid and dashed blue lines in FIG. 14A) were found to be similar. The estimates of relative blood flow measures obtained using the methods of the present disclosure were found to be statistically similar in 10 locations across the thin skull. This indicates that the relative blood flow measures obtained using the methods of the present disclosure are unaffected by the presence of the thin skull. The LSCI estimates (FIG. 14B) however show two significant differences. One, the relative blood flow estimate for region 1 is not close to 0 after the stroke, but is rather close to 0.2 and two, the relative blood flow measures across the boundary (solid and dashed blue lines in FIG. 14B) are different. The first observation is an in vivo reproduction of LSCI's underestimation of large flow changes we reported in an earlier publication, and the second observation is the very limitation that the methods of the present disclosure are designed to overcome. The estimates of relative blood flow measures obtained using the LSCI technique were not found to be statistically similar in 10 locations across the thin skull.

These observations can also be made in relative blood flow measures from the other two pairs of regions, regions 3 & 4 and regions 5 & 6, both in the parenchyma. In these regions a similar trend is seen, but the difference between the two techniques is not as drastic as it is in the blood vessel. Typically, each pixel in the image samples a large distribution of blood flows. The statistical models we use to describe speckle contrast assume that there is one value of blood flow (and hence one τc) in the sampling volume. This assumption is more valid over large blood vessels (or in a microfluidic phantom), where there is a clear direction and rate, for flow. However in the parenchyma, the photons can sample a larger distribution of blood flow rates and a statistical average of these different flow rates is measured. It should be noted that this limitation is common to any dynamic light scattering based measurement. For these reasons, the MESI measurements are likely to be more accurate over the large blood vessel than the parenchyma.

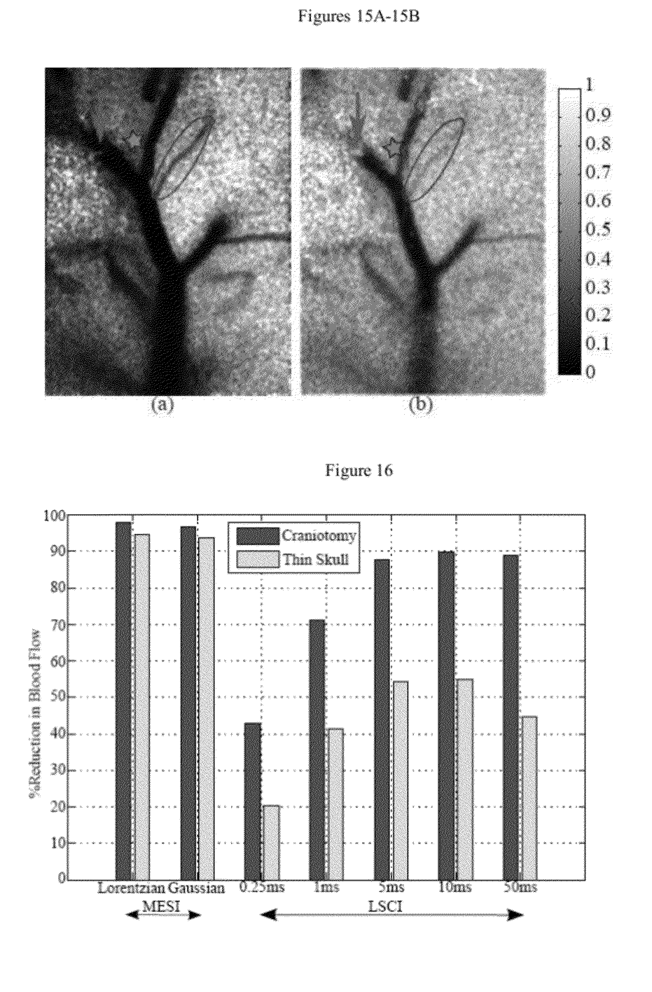

FIG. 15 provides a full field perspective of the relative blood flow changes. These are full field maps of the relative correlation time, computed by taking the ratio of τc under baseline conditions to τc at a single time point after the stroke, as estimated using the methods of the present disclosure (FIG. 15A) and the LSCI technique at 5 ms exposure duration (FIG. 15B). Both images are displayed on a scale of 0 to 1. The thin skull boundary is clearly visible in the LSCI estimate (FIG. 15B), while the demarcation between the craniotomy and the thin skull is less obvious in the MESI estimates (FIG. 15A). This difference is illustrated in the figures using (1) a red arrow and (2) a green star. Additionally, it is seen that some vessels are more visible in the MESI estimate. One example of this is illustrated by the blue oval. These images show that the methods of the present disclosure are better in estimating relative blood flow than LSCI and that these estimates are not affected by the presence of a thin skull. Additionally, the vessels in FIG. 15A appear larger because the blood flow is better resolved using the methods of the present disclosure.

Discussion

The change in the shape of the time integrated speckle variance curves due to the presence of static tissue elements is consistent with previous measurements in flow phantoms. While in the case of the tissue phantoms, the change in the shape was affected in equal parts due to the influence of ρ and vₛ, in the in vivo measures, it was found that the static speckle variance is the more dominant factor. In the microfluidic device used earlier, the flow channel was the only part of the device containing dynamic scatterers. It is believed that in the microfluidic device, the influence of ρ was greater due to the opportunity for a photon to interact with static particles on the sides of the channel and below the channel. This is clearly not the case in vivo, because the only place where a photon can interact from a static particle is from the thin skull. This could explain a comparatively reduced role that ρ plays in the in vivo measurements. Nevertheless, there is no way of accurately determining the value of ρ or vₛ without using Equation 11 and the present disclosure. Hence, the present disclosure provides better suited methods to obtain consistent and accurate measurements of blood flow changes in the presence of a thin skull.

Recently, Duncan et. al. pointed out that a Gaussian function (g1(τ)=e−τ2/τ2c) is a better statistical model to describe the dynamics of ordered flow in a vessel as opposed to the traditionally used negative exponential model [1] (g1(τ)=e−τ/τc). The former corresponds to a Gaussian distribution of velocities, while the negative exponential model corresponds to a Lorentzian distribution of velocities in the sample volume. In order to test this hypothesis, a new MESI expression was derived using the Gaussian function to describe speckle dynamics, and account for scattering from static tissue elements. We substituted g1(τ)=e−τ2/τ2c in Equation 9 and evaluated the integral to arrive at the new expression.

$$K ( T , {τ}_{c} ) = {\{ \begin{matrix}β {ρ}^{2} \frac{{e}^{- 2 {x}^{2}} - 1 + \sqrt{2 π} x erf ( \sqrt{2} x )}{2 {x}^{2}} + \\ 2 β ρ ( 1 - ρ ) \frac{{e}^{- {x}^{2}} - 1 + \sqrt{π} x erf ( x )}{{x}^{2}} + {v}_{ne} + {v}_{noise}\end{matrix} \}}^{1 / 2} \qquad ( 13 )$$

The relative blood flow changes in regions 1 and 2 (FIG. 12A) were estimated using Equation 12 and the methods of the present disclosure. We compared these estimates to those we already obtained using Equation 11 and to the corresponding LSCI estimates at a few exposure durations other than 5 ms. These results are plotted in FIG. 16.

From FIG. 16, it was observed that using the Gaussian statistical model and Equation 13 do not change the estimates of relative flow changes significantly. By incorporating the principles of heterodyne mixing into Equation 13 and by using the methods of the present disclosure, consistent flow measures were still obtained across the boundary of the thin skull. Duncan et. al also pointed out that the differences between the Lorentzian and the Gaussian models are more prominent at the lower exposure durations. By sampling a range of exposure durations, the difference between the two models was minimized. Also as explained earlier, each speckle samples a wide range of flow rates. The differences between the two models are not significant enough to overcome the statistical variability in value of τc. In addition, physiological noise and variability are bigger sources of uncertainty in the fitting process than a small change affected by using a different model. These observations are in agreement with Cheung et. al and Durduran et. al. who showed that the Lorentzian model is a better fit for in vivo blood flow measurements using noninvasive diffuse correlation spectroscopy measurements, due to the complex fluid dynamics of blood flow in vessels.

In FIG. 16, while comparing the LSCI estimates of relative blood flow decrease at multiple exposure durations, it was observed that at 5 ms the percentage reduction in blood flow is about 10% lower than those obtained with the methods of the present disclosure. It was also observed that the choice of exposure time in LSCI can drastically change the estimated blood flow reduction. For example, at 1 ms (another popular choice for in vivo measurements), LSCI predicts a 70% drop in blood flow due to stroke, which is almost 30% lower than the methods of the present disclosure. This is not surprising because the sensitivity to change in blood flow has previously been shown to depend on the choice of exposure duration. This is another reason why the LSCI estimates did not completely pick up the drop in blood flow in a small vessel circled in FIGS. 15A and 15B. It is hence impossible to accurately measure with a single exposure duration, the change in blood flow of all vessels in a field of view that consists of vessels of different diameters (and hence different blood flows). Also, it is noted that the estimates of relative blood flow changes are not consistent across the thin skull boundary for any of the single exposure measurements. From this it can be concluded that for imaging large changes in blood flow or for imaging samples where dynamic and static scatterers are mixed, the methods of the present disclosure are likely to yield more accurate estimates of flow changes.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an”, as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

### REFERENCES

1. Fercher and J. Briers, “Flow visualization by means of single-exposure speckle photography,” Opt. Commun. 37, 326-330 (1981).

2. A. Dunn, H. Bolay, M. Moskowitz, and D. Boas, “Dynamic Imaging of Cerebral Blood Flow Using Laser Speckle,” J. Cereb. Blood Flow Metab. 21, 195-201 (2001).

3. B. Weber, C. Burger, M. Wyss, G. von Schulthess, F. Scheffold, and A. Buck, “Optical imaging of the spatiotemporal dynamics of cerebral blood flow and oxidative metabolism in the rat barrel cortex,” Eur. J. Neurosci. 20, 2664-2670(2004).

4. T. Durduran, M. Burnett, G. Yu, C. Zhou, D. Furuya, A. Yodh, J. Detre, and J. Greenberg, “Spatiotemporal Quantification of Cerebral Blood Flow During Functional Activation in Rat Somatosensory Cortex Using Laser-Speckle Flowmetry,” J. Cereb. Blood Flow Metab. 24, 518-525 (2004).

5. D. Atochin, J. Murciano, Y. Gursoy-Ozdemir, T. Krasik, F. Noda, C. Ayata, A. Dunn, M. Moskowitz, P. Huang, and V. Muzykantov, “Mouse Model of Microembolic Stroke and Reperfusion,” Stroke 35, 2177-2182 (2004).

6. A. Kharlamov, B. Brown, K. Easley, and S. Jones, “Heterogeneous response of cerebral blood flow to hypotension demonstrated by laser speckle imaging flowmetry in rats,” Neurosci. Lett 368, 151-156 (2004).

7. K. Murari, N. Li, A. Rege, X. Jia, A. All, and N. Thakor, “Contrast-enhanced imaging of cerebral vasculature with laser speckle,” Appl. Opt. 46, 5340-5346 (2007).

8. B. Ruth, “Measuring the steady-state value and the dynamics of the skin blood flow using the non-contact laser speckle method.” Med. Eng. Phys. 16,105-11 (1994).

9. K. Forrester, C. Stewart, J. Tulip, C. Leonard, and R. Bray, “Comparison of laser speckle and laser Doppler perfusion imaging: Measurement in human skin and rabbit articular tissue,” Med. Biol. Engg. Comp. 40, 687-697 (2002).

10. B. Choi, N. Kang, and J. Nelson, “Laser speckle imaging for monitoring blood flow dynamics in the in vivo rodent dorsal skin fold model,” Microvasc. Res 68, 143-146 (2004).

11. K. Yaoeda, M. Shirakashi, S. Funaki, H. Funaki, T. Nakatsue, A. Fukushima, and H. Abe, “Measurement of microcirculation in optic nerve head by laser speckle flowgraphy in normal volunteers,” Am. J. Opthalmol. 130, 606-610(2000).

12. C. Ayata, A. Dunn, Y. Gursoy-Ozdemir, Z. Huang, D. Boas, and M. Moskowitz, “Laser speckle flowmetry for the study of cerebrovascular physiology in normal and ischemic mouse cortex,” J. Cereb. Blood Flow Metab. 24, 744-755 (2004).

13. A. Strong, E. Bezzina, P. Anderson, M. Boutelle, S. Hopwood, and A. Dunn, “Evaluation of laser speckle flowmetry for imaging cortical perfusion in experimental stroke studies: quantitation of perfusion and detection of periinfarct depolarisations,” J. Cereb. Blood Flow Metab. 26, 645-53 (2006).

14. A. Dunn, A. Devor, A. Dale, and D. Boas, “Spatial extent of oxygen metabolism and hemodynamic changes during functional activation of the rat somatosensory cortex,” NeuroImage 27, 279-290 (2005).

15. S. Yuan, A. Devor, D. Boas, and A. Dunn, “Determination of optimal exposure time for imaging of blood flow changes with laser speckle contrast imaging,” Appl. Opt. 44, 1823-1830 (2005).

16. M. Stern, “In vivo evaluation of microcirculation by coherent light scattering,” Nature (London) 254, 56-8 (1975).

17. H. Shin, P. Jones, M. Garcia-Alloza, L. Borrelli, S. Greenberg, B. Bacskai, M. Frosch, B. Hyman, M. Moskowitz, and C. Ayata, “Age-dependent cerebrovascular dysfunction in a transgenic mouse model of cerebral amyloid angiopathy,” Brain 130, 2310-2319 (2007).

18. R. Bandyopadhyay, A. Gittings, S. Suh, P. Dixon, and D. Durian, “Speckle-visibility spectroscopy: A tool to study time-varying dynamics,” Rev. Sci. Instrum. 76, 093110 (2005).

19. B. Berne and R. Pecora, Dynamic Light Scattering : with Applications to Chemistry, Biology and Physics (Dover Publications, 2000).

20. R. Bonner and R. Nossal, “Model for laser Doppler measurements of blood flow in tissue,” Appl. Opt. 20, 2097-2107(1981).

21. K. Forrester, J. Tulip, C. Leonard, C. Stewart, and R. Bray, “A laser speckle imaging technique for measuring tissue perfusion,” IEEE Trans. Biomed. Engg. 51, 2074-2084 (2004).

22. P. Dixon and D. Durian, “Speckle Visibility Spectroscopy and Variable Granular Fluidization,” Phys. Rev. Lett. 90, 184302 (2003).

23. P. Lemieux and D. Durian, “Investigating non-Gaussian scattering processes by using n^{th}-order intensity correlation functions,” J. Opt. Soc. Am. A 16, 1651-1664 (1999).

24. H. Cheng, Q. Luo, S. Zeng, S. Chen, J. Cen, and H. Gong, “Modified laser speckle imaging method with improved spatial resolution,” J. Biomed. Opt. 8, 559-564 (2003).

25. P. Li, S. Ni, L. Zhang, S. Zeng, and Q. Luo, “Imaging cerebral, blood flow through the intact rat skull with temporal laser speckle imaging,” Opt. Lett. 31, 1824-1826 (2006).

26. P. Zakharov, A. Völker, A. Buck, B. Weber, and F. Scheffold, “Quantitative modeling of laser speckle imaging,” Opt. Lett. 31, 3465-3467 (2006).

27. D. Boas and A. Yodh, “Spatially varying dynamical properties of turbid media probed with diffusing temporal light correlation,” J. Opt. Soc. Am. A 14, 192-215 (1997).

28. J. Anderson, D. Chiu, R. Jackman, O. Chemiayskaya, J. McDonald, H. Wu, S. Whitesides, and G. Whitesides, “Fabrication of Topologically Complex Three-Dimensional Microfluidic Systems in PDMS by Rapid Prototyping,” Science 261, 895 (1993).

29. A. Oldenburg, F. Toublan, K. Suslick, A. Wei, and S. Boppart, “Magnetomotive contrast for in vivo optical coherence tomography,” Opt. Express 13, 6597-6614 (2005).

What is claimed is:

1. A method for quantitative blood flow imaging comprising computing a quantitative blood flow image from a speckle pattern using the following equation:
$$K ( T , {τ}_{c} ) = {\{ β {ρ}^{2} \frac{{e}^{- 2 x} - 1 + 2 x}{2 {x}^{2}} + 4 β ρ ( 1 - ρ ) \frac{{e}^{- x} - 1 + x}{{x}^{2}} + {v}_{ne} + {v}_{noise} \}}^{1 / 2} , \\ where x = \frac{T}{{τ}_{c}} , ρ = \frac{{I}_{f}}{( {I}_{f} + {I}_{s} )}$$
is the fraction of total light that is dynamically scattered, β is a normalization factor to account for speckle averaging effects, T is the camera exposure duration, τ_{c} is the correlation time of the speckles, vₙₒᵢₛₑ is the constant variance due to experimental noise and vₙₑ is the constant variance due to nonergodic light.

2. The method of claim 1 wherein the quantitative blood flow imaging is conducted in the presence of a static scatter.

3. The method of claim 2 wherein the static scatter is bone.

4. A method for quantitative blood flow imaging comprising:
providing a system comprising:
a laser light source;
a light modulator; and
a detector for the measurement of reflected light comprising at least one camera, at least one magnification objective, and at least one microprocessor or data acquisition unit;
illuminating a sample and detecting a speckle pattern using the system; and
computing a quantitative blood flow image using the following equation:
$$K ( T , {τ}_{c} ) = {\{ β {ρ}^{2} \frac{{e}^{- 2 x} - 1 + 2 x}{2 {x}^{2}} + 4 β ρ ( 1 - ρ ) \frac{{e}^{- x} - 1 + x}{{x}^{2}} + {v}_{ne} + {v}_{noise} \}}^{1 / 2} , \\ where x = \frac{T}{{τ}_{c}} , ρ = \frac{{I}_{f}}{( {I}_{f} + {I}_{s} )}$$
is the fraction of total light that is dynamically scattered, β is a normalization factor to account for speckle averaging effects, T is the camera exposure duration, τ_{c} is the correlation time of the speckles, vₙₒᵢₛₑ is the constant variance due to experimental noise and vₙₑ is the constant variance due to nonergodic light.

5. The method of claim 4 wherein quantitative blood flow imaging is conducted in the presence of a static scatter.

6. The method of claim 5 wherein the static scatter is bone.

7. The method of claim 4 wherein the system is automated, semi-automated, or both.

8. The method of claim 4 wherein the detector comprises a plurality of cameras.

9. The method of claim 4 wherein the detector detects reflected light.

10. The method of claim 4 wherein the laser light source is pulsed to create multiple exposures.

11. The method of claim 4 wherein the light modulator varies the intensity of the laser light source.

12. The method of claim 4 wherein the light modulator is an acousto-optic modulator, an electro-optic modulator, or a spatial light modulator.

13. A method of measuring blood velocity in a tissue comprising:
illuminating a tissue surface with coherent light from a laser light source;
receiving reflected and scattered coherent light from the tissue on a photodetector;
obtaining a speckle pattern from the reflected and scattered coherent light;
computing a quantitative blood flow image using the speckle pattern and the following equation:
$$K ( T , {τ}_{c} ) = {\{ β {ρ}^{2} \frac{{e}^{- 2 x} - 1 + 2 x}{2 {x}^{2}} + 4 β ρ ( 1 - ρ ) \frac{{e}^{- x} - 1 + x}{{x}^{2}} + {v}_{ne} + {v}_{noise} \}}^{1 / 2} , \\ where x = \frac{T}{{τ}_{c}} , ρ = \frac{{I}_{f}}{( {I}_{f} + {I}_{s} )}$$
is the fraction of total light that is dynamically scattered, β is a normalization factor to account for speckle averaging effects, T is the camera exposure duration, τ_{c} is the correlation time of the speckles, vₙₒᵢₛₑ is the constant variance due to experimental noise and vₙₑ is the constant variance due to nonergodic light.

14. The method of claim 13 further comprising evaluating the quantitative blood flow image and thereby determining blood velocity and perfusion in the tissue.

15. A multi-exposure laser speckle contrast imaging system comprising:
a laser light source;
a light modulator;
a detector for the measurement of reflected light comprising at least one camera and at least one magnification objective;
a microprocessor or data acquisition unit; and
a memory, the memory including executable instructions that, when executed, cause the microprocessor or data acquisition unit to compute a quantitative blood flow image using the following equation:
$$K ( T , {τ}_{c} ) = {\{ β {ρ}^{2} \frac{{e}^{- 2 x} - 1 + 2 x}{2 {x}^{2}} + 4 β ρ ( 1 - ρ ) \frac{{e}^{- x} - 1 + x}{{x}^{2}} + {v}_{ne} + {v}_{noise} \}}^{1 / 2} , \\ where x = \frac{T}{{τ}_{c}} , ρ = \frac{{I}_{f}}{( {I}_{f} + {I}_{s} )}$$
is the fraction of total light that is dynamically scattered, β is a normalization factor to account for speckle averaging effects, T is camera exposure duration, τ_{c} is correlation time of the speckles, vₙₒᵢₛₑ is a constant variance due to experimental noise and vₙₑ is a constant variance due to nonergodic light.
